(19) United States
(12) Patent Application Publication
(10) Pub. No.: US 2025/0098945 A1
(43) Pub. Date: Mar. 27, 2025

(54) ENDOSCOPE WITH A BENDING SECTION CONTROLLED BY STEERING WIRES

(71) Applicant: AMBU A/S, Ballerup (DK)

(72) Inventor: Frederik Clausager Vemb HANSEN, Copenhagen NV (DK)

(73) Assignee: AMBU A/S, Ballerup (DK)

(21) Appl. No.: 18/895,971

(22) Filed: Sep. 25, 2024

(30) Foreign Application Priority Data
Sep. 26, 2023 (EP) 23199852.7

(51) Int. Cl. A61B 1/005

(52) U.S. Cl. CPC A61B 1/0052; A61B 1/0057

(57) ABSTRACT

An endoscope (1) having a bending section (20) including segments (22) connected to each other by hinges (24). The bending section has a central passage (12) passing each segment (22) and extends from a distal end to a proximal end, where an inner surface is facing and encircling the central passage (12). At least two grooves (18) into the inner surface extend in parallel with the central passage (12) and in open connection with the central passage. Two steering wires (25) pass in the grooves through the bending section. An anchor (15, 15′, 15″) is provided with a holding part (17, 17′) such that displacement of the steering wires (25) from the grooves (18) towards a center of the central passage (12) is blocked.

### CROSS-REFERENCE TO RELATED APPLICATIONS

This application claims priority from and the benefit of European Patent Application No. 23 199 852.7, filed Sep. 26, 2023; the disclosure of said application is incorporated by reference herein in its entirety.

### TECHNICAL FIELD

The present disclosure relates to an endoscope comprising an insertion cord including a bending section. Bending of the bending section is controlled by steering wires running in dedicated parts of the bending section.

### BACKGROUND

Flexible endoscopes for medical purposes are often provided with a bending section at a distal end of an insertion tube. The bending section enables the user of the endoscope to maneuver the distal tip of the endoscope inside the human anatomy, such as in the airways, in the kidneys or the gastro-intestinal system, and e.g., to study or perform procedures at tissue of interest. The bending section is typically bent by pulling steering wires. If the endoscope is a 2-way bending endoscope, i.e., bending in two opposite directions but in the same plane, the bending section will typically be controlled by two steering wires, which are controlled by one steering wire actuator arranged in the handle of the endoscope, allowing the user to bend the bending section by adjusting a bending lever. If the endoscope is a four-way bending endoscope, i.e., also bending in two opposite directions in a second plane perpendicular to the first mentioned plane, the bending section will typically be controlled by four steering wires, where one steering wire actuator controls two steering wires for bending in the first plane, and the other steering wire actuator controls the two other steering wires for bending in the second plane. Typically, each steering wire actuator is controlled by a rotational wheel on the endoscope handle.

The steering wires will pass from the handle to the proximal end of the bending section inside wire pipes, which are tubes having limited compressibility. Thereby so-called Bowden cables are formed. The wire pipes are at their proximal end connected inside the handle. At the distal end of the wire pipes, they are connected to the proximal end of the bending section, whereas the steering wires continue through the bending section. The wire pipes are held stationary and translatory movement of the steering wire at the proximal end is transmitted to the distal end as a corresponding translatory movement of the steering wire relative to the wire pipe. The steering wires may translate in closed lumens of the bending section.

For single use endoscopes the bending section may be molded in one piece from a polymer material. Especially for narrow bending sections for endoscopes with a small outer diameter, this molding process can be difficult. E.g., for ureteroscopes, often having a relatively long bending section and an outer diameter of maybe less than 3 mm, it may be difficult to manufacture, and find space for, closed lumens for the steering wires. The long and thin cores needed to mold closed steering wire lumens in the molding process will be fragile and could cause frequent interruptions of the molding process.

It is simpler to manufacture a bending section with grooves or recesses functioning as open channels for the steering wires. Such grooves are in open connection with the rest of the lumen. However, this design of the bending section does involve a minor risk that the steering wire may be displaced from the dedicated open channel during some procedures. For example, a procedure involving specific maneuvering inside the kidney. During such procedure, maneuvering the proximal end of the bending section may be bent in one direction while the operator of the endoscope manipulates the steering wire actuator to bend the distal end of the bending section in the opposite direction.

### SUMMARY

It is an object of the present disclosure to prevent displacement of the steering wire out of the open channel or groove. In a first aspect, the object is achieved by a bending section comprising an anchor that prevents such displacement. In a second aspect, the object is achieved by an endoscope comprising said bending section and said anchor. In a third aspect, the object is achieved by a method of making the endoscope according to the second aspect.

In one embodiment according to the first aspect, the bending section comprises an inner, or central, lumen extending in a longitudinal direction. Grooves extend from a periphery of the inner lumen. The anchor comprises a holding part to block displacement of steering wires from the grooves towards the central passage.

One advantage of the anchor is that it enables molding of a small outer diameter bending section, molded in one piece and with the central lumen, while reducing or eliminating the risk of displacement of the steering wires during endoscopic procedures.

The bending section may comprise segments connected to each other by hinges and molded in one piece therewith. The central passage, or inner lumen, passes each segment and extends from a distal end to a proximal end of the bending section, where an inner surface of a circumferential wall of one or more of the segments faces and encircles the central passage. The grooves extend in parallel with, and in open connection with, the central passage. The grooves may be cut-outs in the circumferential walls of the segments.

The hinges allow two neighboring segments to bend relative to each other in a bending plane. The segments and the hinges form an outer surface of the bending section. By neighboring it is meant that the distal surface of a segment faces the proximal surface of its neighbor or adjacent segment (or vise versa) so that the two segments are next to each other in a sequence. By bending plane it is meant a plane that bends with the bending section. Thus, the bending plane traverses the hinges. When the bending section is straight, the bending plane is also straight.

An indent may extend from the outer surface into at least one segment or in at least one hinge. Further, the anchor may be arranged in the indent.

In a variation of the present embodiment, the anchors are placed in at least two indents in positions in segments, or next to segments, placed in only the first 60% of the bending section, i.e., the 60% of the bending section length starting from the proximal end of the bending section and extending toward the distal end of the bending section. Placing the anchors in this first proximal part of the bending section is advantageous, since it has been found that the force on the steering wires in the direction towards a center of the central passage of the bending section is largest in this first proximal part. In general, it is also preferred to keep the total number of anchors low to keep the manufacturing costs as low as possible. Preferably, anchors should be applied in, or in connection with, less than half of the segments. Preferably, anchors should be applied in, or in connection with, less than a third of the segments. In a further variation, anchors are arranged in at least two segments, and in less than 40% of the segments, alternatively in less than 30% of the segments. In one example, anchors are arranged in between 10% and 30% of the segments within the proximal section of the bending section, the proximal section comprising less than 60% of the length of the bending section. The anchors may be spaced so that there is at least one segment without anchors between segments with anchors.

In a variation of the present embodiment, at least two segments are provided with anchors such that displacement of the two steering wires is blocked. Having the anchors within the segments provides a very stable positioning of the anchors.

In a variation of the present embodiment, at least two segments provided with anchors are separated by at least one segment without an anchor. This arrangement, to have the anchors spread out and not in neighboring segments, has been found to provide a good trade-off between manufacturing costs and stability of the bending section with respect to the steering wires not being displaced. In a further variation, there may be at least two segments provided with anchors which are separated by at least two segments without an anchor.

In a variation of the present embodiment, each anchor has a holding part extending into a space where the groove and the central passage meet. The holding part has the advantage that it provides a direct blockage preventing displacement of the steering wire.

In a variation of the present embodiment, the anchors have an exterior surface being level with an outer surface of a segment and the anchors have an interior surface being level with the inner surface. This enables that the anchor may have sufficient dimensions to provide sufficient mechanical strength to resist the forces from the steering wire, e.g., via the coiled spring.

In a variation of the present embodiment, the anchors are made from hardened glue. This enables a simple and cost-effective manufacturing of the endoscope and has also been found to result in a very stable and efficient anchor. In a further variation each anchor is adhered to a segment of the bending section.

In a variation of the present embodiment, a ledge extends from the segment between the indent and the central passage, and the ledge supports the anchor. This provides a strong and stable support for, especially, the glued anchor variation.

In a variation of the present embodiment, the anchors are made from metal discs. This provides anchors which takes up a minimum amount of space in the proximal to distal length direction of the bending section.

In one variation, an anchor arranged in connection with a segment may be able to keep both steering wires inside the grooves.

In another variation, an anchor in a segment may only keep one steering wire in the corresponding groove. Two anchors in the same segment may be preferred, but anchors for the two different steering wires could also be placed in different segments.

Segments of the bending section according to the present embodiment and variations thereof include a proximal end segment, a distal end segment and intermediate segments arranged between the proximal end segment and the distal end segment. The segments are not necessarily identical. Especially, the proximal end segment and the distal end segment may have features enabling the connection of the bending section to an insertion tube of the insertion cord and to a distal tip housing, respectively.

In one embodiment according to the second aspect, the endoscope comprises a bending section according to the first aspect and variations thereof, and steering wires arranged in the grooves of the bending section.

The endoscope may comprise a handle and an insertion cord extending from the handle and including the bending section, and two steering wires extending from the handle to the distal end of the bending section. Each steering wire passes in a respective one of the grooves through the bending section.

In a variation of the present embodiment, the steering wires in the bending section are passing inside coil springs, the coil springs with the steering wires are each arranged in a groove, and the anchor is arranged to block displacement of the coil springs, and thereby the steering wires, from the grooves towards the center of the central passage. This reduces the risk of the steering wires moving into the narrow space between a working channel and the inner surface of the bending section, especially in parts of the bending section where there are no anchors.

In one embodiment according to the third aspect, the method comprises providing a bending section according to the first aspect; arranging two steering wires to extend from the handle to the distal end of the bending section, such that each steering wire passes in a separate groove through the bending section; and arranging an anchor in the indent, where the anchor is provided with a holding part such that displacement of the steering wires from the grooves towards a center of the central passage is blocked.

In another embodiment according to the third aspect, the method comprises: providing a bending section molded in one piece from a fused polymer, and having segments connected to each other by hinges, the hinges allowing two neighboring segments to bend relative to each other in a bending plane, the segments and the hinges forming an outer surface of the bending section, a central passage passing each segment and extending from a distal end to a proximal end of the bending section, where each segment has a circumferential wall, the circumferential wall has an inner surface facing and encircling the central passage, at least two grooves extending from the inner surface into the circumferential wall, the grooves extending in parallel with the central passage and in open connection with the central passage, the central passage and the grooves extending through the segments of the bending section, an indent extending from the outer surface into at least one segment or in at least one hinge; arranging two steering wires to extend from the handle to the distal end of the bending section, such that each steering wire passes in a separate groove through the bending section; arranging an anchor in the indent, where the anchor is provided with a holding part such that displacement of the steering wires from the grooves towards a center of the central passage is blocked.

The embodiments of the method according to the third aspect have been found to be cost-effective ways to manufacture bending sections for endoscopes, e.g., single use endoscopes, having a small diameter insertion cord. The outer diameter of the insertion cord, including the bending section, may be 3 mm or smaller.

In a fourth aspect, the disclosure relates to a system comprising an endoscope according to the second aspect and variations thereof, a monitor and a control unit.

The expression “distal” is defined to be in the direction toward the patient, and “proximal” is defined to be in the direction away from the patient. For the handle of the endoscope, the distal end will be the end where the insertion tube is connected, and the proximal end is the opposite end. Further, the expression “handle” may be a positioning interface, or interface, which functions to control the position of the insertion cord and operating the bending section. The handle, or positioning interface, may be an interface operated by a robotic arm, or it may be a handle operated by the hand of an endoscope user.

One steering wire is counted as one passage from the steering wire actuator (or roller) to the distal end of the bending section. I.e., if the same unbroken steering wire continues from the steering wire actuator to the distal end of the bending section and back to the steering wire actuator, and one part is applied for bending for example to one side and the other part is applied for bending to the opposite side, this is counted as two steering wires, e.g., first and second steering wires.

### BRIEF DESCRIPTION OF THE FIGURES

The above-mentioned embodiments and variations, features and advantages thereof will be further elucidated by the following illustrative and nonlimiting detailed description of embodiments disclosed herein with reference to the appended drawings, wherein:

FIG. 1 shows an endoscope and a monitor with a control unit.

FIG. 2 shows an example of a steering wire actuator, here in the form of a roller, and a wire pipe fastener.

FIG. 3 shows a distal end of an endoscope including a bending section.

FIG. 4 shows a cross-sectional view of a bending section provided with a glued anchor.

FIG. 5 shows a part of a bending section where one segment is provided with anchor.

FIG. 6 shows the bending section of FIG. 5 from a different angle.

FIG. 6A shows an insert anchor.

FIG. 6B shows a cylinder.

FIG. 6C shows a bending section with anchors.

FIG. 7 shows a part of a bending section with a disc anchor.

FIG. 8 shows an example of a separate disc anchor.

FIG. 9 shows a cut through bending section with the disc anchor from FIG. 8.

FIG. 10 shows a part of a bending section with a different disc anchor.

FIG. 11 shows a part of a bending section with part of segments removed to give a better view of the disc anchor.

FIG. 12 shows a cross-sectional view of a bending section provided with the disc anchor of FIG. 10.

FIG. 13 shows the disc anchor included in FIGS. 10-12.

FIG. 14 schematically illustrates S-shaped curvature of a bending section.

In the drawings, corresponding reference characters indicate corresponding parts, functions, and features throughout the several views. The drawings are not necessarily to scale and certain features may be exaggerated in order to better illustrate and explain the disclosed embodiments. For simplicity, this disclosure will focus on a two-way bending endoscope, but the disclosure is relevant for, and covers, also a four-way bending endoscope.

### DETAILED DESCRIPTION

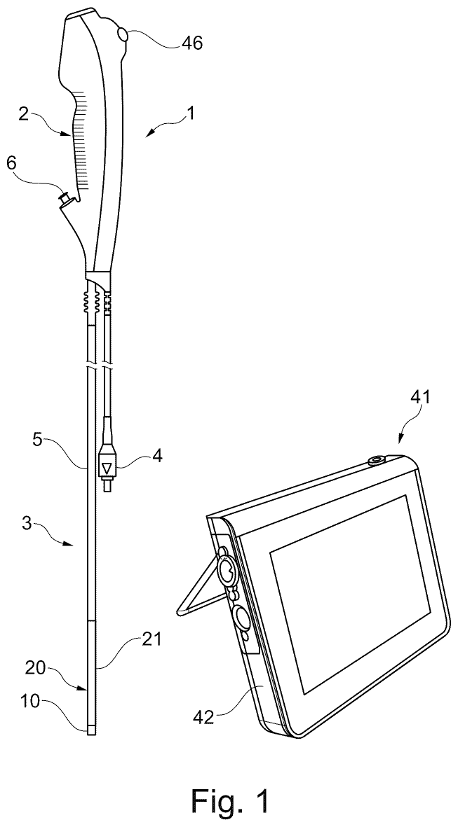

FIG. 1 illustrates an endoscope 1, which comprises a handle 2, an insertion cord 3 and an electrical cable with a connector 4 for connecting the endoscope 1 to a monitor 41. The insertion cord 3 is the part to be inserted into a body lumen during an endoscopic procedure. The insertion cord comprises a distal tip 10, a bending section 20 and a main tube 5. The handle 2 may comprise an entrance to a working channel 6 running through the insertion cord to the distal tip. The handle also comprises a bending lever 46, which can be used for bending the bending section. The bending section may include a bending cover 21.

The distal tip 10 comprises a camera and light emitters, e.g., in the form of one or more LEDs or the end of an optical light fiber, enclosed in a tip housing. Examples of distal tips 10 are disclosed in commonly owned U.S. Pat. No. 11,291,352; the disclosure of said patent is incorporated by reference herein in its entirety. The endoscope may consist of an ureteroscope or a cystoscope, comprising a working channel tube in a central passage, the distal tip including the camera, an outer diameter of the bending section being between 2.0 and 3.0 mm, with a cross-sectional area of the working channel tube being at least 40% of a cross-sectional area of the bending section. The shape of the bending section as described below enables such a large working channel tube relative to such a small bending section diameter, which is advantageous for specialized procedures performed with ureteroscopes and cystoscopes and results in some of the problems solved by the anchors.

The monitor 41 may be combined with an electronic circuit for receiving and processing the image stream from the camera as well as a processor for image processing, user interface, storage of images etc. But the monitor and the electronic circuit and processor may also be separate parts. The electronic circuit and the processor part are also referred to as a control unit 42.

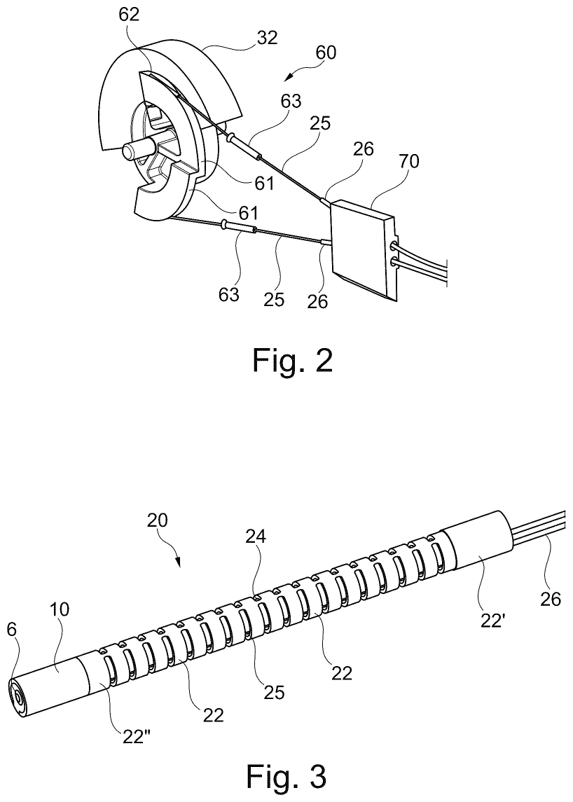

FIG. 2 shows a schematic example of a roller 32 for a steering wire actuator 60 configured for bending the bending section by pulling the steering wires 25. The steering wires 25 are moved by rotation of wire drum curved surfaces 61, which are a circular arc surfaces or a curved surface placed on the roller 32 and supporting the steering wires. In the example, one end of the steering wire has been drawn through a fixing structure 62 and bent back and fastened to itself by a crimp 63. The proximal ends of the wire pipes 26 are fixated in the wire pipe fastener 70, whereas the distal ends of the wire pipes are secured to the proximal end of the bending section 20. Both the wire pipe fastener 70 and the roller 32 are often secured in the handle 2. The system in FIG. 2 is for a two-way bending endoscope.

FIG. 3 shows a distal end of the insertion cord 3 of an endoscope, where the bending cover (not shown) typically covering the bending section 20 and often making this part watertight, has been removed. The proximal end of the bending section is attached to the main tube 5, and the distal end of the bending section is attached to the distal tip 10. In this example the bending section is molded in one piece and comprises a number of segments 22 and hinges 24. A proximal end segment 22′ is connected to the distal end of the main tube 5. A distal end segment 22″ is connected to the distal tip 10. The segments are held together by the hinges 24, so that the segments can be bent relative to each other by manipulation of the steering wires 25. An example of such a bending section molded in one piece can be seen in commonly owned U.S. Pat. No. 11,937,781; the disclosure of said patent is incorporated by reference herein in its entirety.

Alternatively, the bending section 20 could be extruded in a relatively soft and resilient material, e.g., a foam-like material, with lumens for steering wires, electrical wires and the tubes passing through.

Steering wires 25 are connected in a fixed connection to the distal end, e.g., the distal end segment 22″. I.e., the steering wire is preferably not movable in relation to the distal end segment. Between the handle and the distal end of the main tube 5, or the proximal end of the bending section 20, the steering wire 25 is guided in a wire pipe 26 which is secured in the handle 2 distal to the steering wire actuator 60. Other designs of the bending section are also possible.

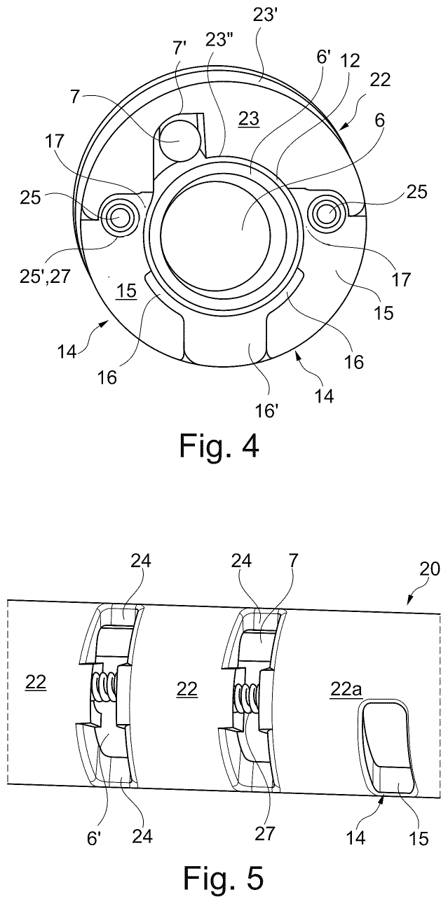

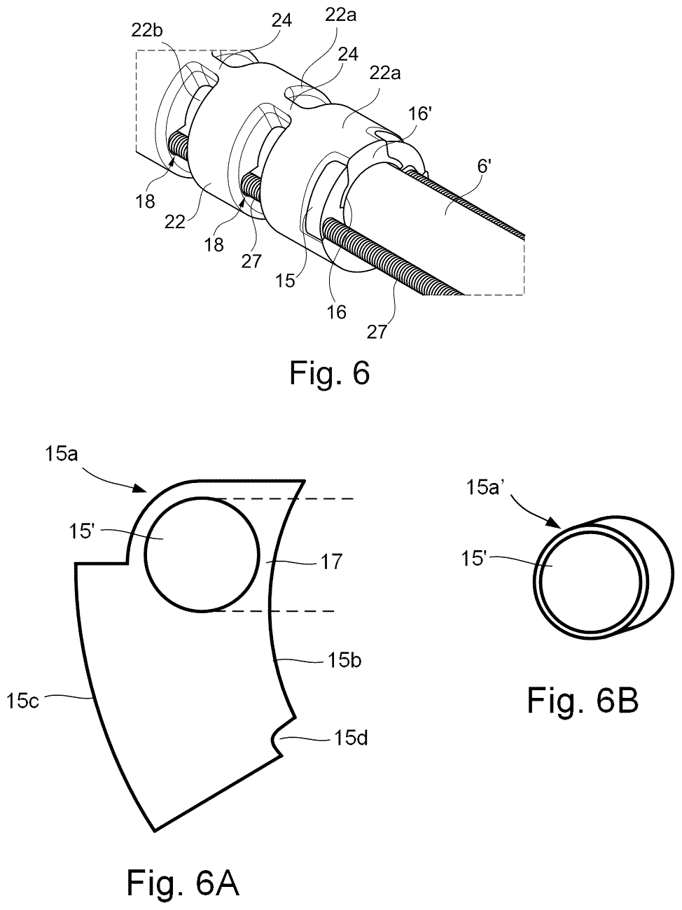

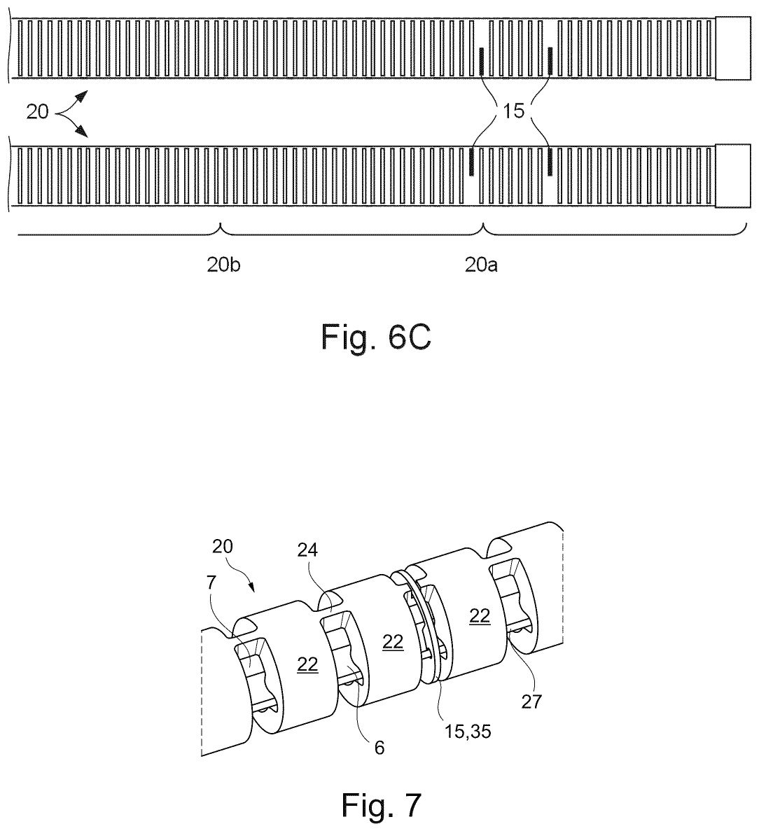

FIGS. 4-6 show an embodiment of a bending section 20 according to the first aspect, where the bending section 20 is provided with only one inner lumen or central passage 12. This central passage 12 has grooves or recesses 18 (shown in FIG. 6) in open connection with the rest of the central passage 12. The steering wires 25 are arranged in these grooves 18. An anchor 15 is provided to avoid that the steering wire 25 is displaced from, or leaves, the groove 18, during bending operations. The cross-sectional view in FIG. 4 is made at the position of the glued anchor 15, and the indent 14, which is best seen in FIG. 5. The glue anchor 15 comprises a holding part 17 positioned between the groove 18 and the central passage 12.

The segment 22 comprises a circumferential wall 23 extending from an outer surface 23′ to an inner surface 23″. The central passage 12, an electrical wire bundle groove 7′, and the steering wire grooves 18 define, at least in part, the inner surface pf the circumferential wall 23. The electrical wire bundle groove 7′ and the steering wire grooves 18 can be said to be cut-outs in the wall 23 extending from the periphery of the central passage 12. As shown, the central passage 12 has a circular shape. Other shapes, such as oblong, are also permissible. A working channel tube 6′ is positioned in the central passage 12 and comprises at least a portion of the working channel 6. An electrical wire bundle 7 is positioned in the electrical wire bundle groove 7′. The steering wires 25 may be arranged inside coil springs 27. A ledge 16 supports the anchors 15, as described below.

The glue anchor 15 shown in FIGS. 4-6 may be made from a hardened adhesive which fills an indent 14 made in a segment 22. FIG. 4 shows a cross-sectional view of a segment 22, where two indents 14 are made. The indents have been filled with a glue or adhesive, which have been hardened, and thereby form the anchors 15. FIG. 5 shows the bending section 20 from one side, whereby one indent 14 in the segment 22 can be seen. The anchor 15, made from a hardened transparent adhesive/glue, is placed in this indent 14. FIG. 4 shows that a similar anchor 15 may be placed at two opposite sides of the bending section 20, one anchor for each steering wire 25.

The anchors 15 have a hole 15′ and a holding part 17 extending from the hole 15′ into the space between the steering wire 25 and the central passage 12. These holding parts 17 of the anchors prevent displacement of the steering wires 25 in the direction toward the central passage 12. The holding parts 17 can completely or partially surround the coil springs 27. If partially, they should surround enough of the periphery of the coil springs 27 so that any gap is smaller than the diameter of a coil spring 27. The hole 15′ receives or is formed around the steering wire 25 and, if present, the coil spring 27.

It is shown in FIGS. 4-6 that the steering wires in the bending section may be arranged inside the coil springs 27. The coil springs 27 with the steering wires 25 are each arranged in a groove 18, and the anchor 15 is arranged to block displacement of the coil springs 27, and thereby the steering wires 25, from the grooves 18 towards a center of the central passage 12. This reduces the risk of the steering wires moving into the narrow space between the working channel 6 and the inner surface 23″ of the bending section 20. This may be advantageous for the segments 22 where no anchors 15 are arranged. If the springs 27 are part of the design, the holding part 17 of the anchors will extend into the space between the springs 27 and the working channel 6.

The use of springs 27 for guiding or enclosing the steering wires through a bending section where grooves 18, i.e., the dedicated lumen for steering wires, are in open connection to the lumen for the working channel, is described further in commonly owned U.S. Patent Publication No. 2022/0233054A1; the disclosure of said publication is incorporated by reference herein in its entirety, and works for a two-way bending endoscope.

The spring 27 is made from a wound wire, e.g., a steel wire. The distance between two neighboring windings of the spring 27 may be referred to as the pitch of the spring 27. So, the minimum pitch is similar to the diameter of the wire used for the spring. The pitch should be selected so that the springs can both be compressed and extended during bending of the bending section. The pitch may also vary, so that it is low in segments where an anchor 15 is placed, and higher in other positions. A low pitch may prevent liquid glue from entering between the windings and adhere to the steering wire during the gluing process providing a glued anchor. The pitch may be at a minimum in places where an anchor is to be placed. However, if the liquid glue has a high viscosity, the pitch may be larger than the minimum and still prevent the glue from getting in contact with the steering wire.

The steering wires 25 may, alternatively, be moveably arranged through the anchors 15 without the springs 27. In that case, it must be ensured that there is no adherence between the steering wire and the anchors in manufacturing. This can be ensured by applying a lubricant to the steering wire 25. This may be applied before application and hardening of the glue. The glue and the steering wire material may also be selected so that there will be no adherence. In that case the lubricant might not be necessary but may still be advantageous for lowering the friction between steering wire and hardened glue.

In another variation, an insert anchor 15a is placed in the indent. The insert anchor 15a has a hole 15′ for the steering wire and may comprise a portion shaped to fit in the groove. The insert anchor 15a may have the same shape as the glue anchor 15. As shown in FIG. 6A, the insert anchor 15a has the hole 15′, the holding part 17 (shown between dashed lines), an inner surface 15b supporting the working channel tube, and an outer surface potentially supported by the bending cover. A cutout 15d extending from the inner surface 15b is configured to match a corner of the ledge 16. Thus, the insert anchor 15a may be placed in an indent 14 located between the proximal and distal surfaces 22a, 22b of a segment, or may be placed in an indent 14 located at the proximal end or the distal end of a segment, by translating the insert anchor 15a into the indent 14. The shape of the insert anchor 15a is configured to support itself, fully or mostly mechanically, in the indent 14, although glue can assist. The adhesive may be avoided, if the press fit and the bending cover hold the insert in place, particularly since the steering wire prevents the insert from falling out of the indent.

In a further variation shown in FIG. 6B, small cylinders 15a′ are placed in the indents and the steering wires are threaded through the small cylinders. The glue or adhesive bonds to the small cylinders instead of the wire coils 27 or the steering wires 25. The cylinders may be made of metal and may comprise a textured surface or a chemically treated surface to aid in such adhesive bonding.

Therefore, as described above, the holding part 17 may be comprised of a solidified adhesive surrounding the steering wire or the coil or the small cylinder directly, or an insert anchor.

The anchors 15 shown in FIGS. 4-6 are relatively large compared to the size of the holding part 17 actively holding the steering wire in the grooves 18. One reason for this is that bending sections 20 molded in one piece from a polymer material are often made from Polyoxymethylene (POM), and most glues do not adhere very well to POM. Therefore, the geometry of the anchor 15, made from hardened glue, may be such that the anchor is kept in the correct position in the indent 14 of the segment 22, also if the glue does not adhere to the bending section material. This may be achieved by a ledge or wing 16 extending between part of the anchor 15 and the central passage 12. The ledge 16 may be formed in one piece with the segment and the rest of the bending section and may be molded from the same fused polymer material. The ledge 16 can thus support the anchor 15 and is adapted to keep the anchor 15 in the correct position. As shown, ledges 16 are connected to a central ledge portion 16′ and together therewith provide part of the inner surface 23″ of the wall 23 and the circumference of the central passage 12. The ledges 16 are thin relative to the central portion 16′, whereby the central portion 16′ extends from the inner surface 23″ to the outer surface 23′ and the wings 16 extend from the inner surface 23″ to somewhere intermediate the inner surface 23″ and the outer surface 23′. In another example, the central portion 16′ does not extend to the outer surface 23′ and the two anchors 15 may connect each other, potentially allowing insertion of glue or adhesive at a single point. The glue anchors may fill the indent to form the holding part 17. The glue anchors may also fill the grooves 18 to form a holding part 17 that extends longitudinally from the indent into the groove. This provides an even stronger holding part. The glue does not need to completely fill the space between the spring coil and the central channel, it suffices that the holding part is sturdy enough to retain the spring coil and/or steering wire at the particular longitudinal position of the bending section where it is placed, Thus, the holding parts can differ in shape. The wall of a segment has a proximal surface and a distal surface, and the indent is positioned between the proximal surface and the distal surface. A surface of the ledge may form part of the proximal or distal surface of the segment, the indent extending inwardly from such surface so that the indent is positioned between the proximal surface and the distal surface. The indent can also be a cut-out in the wall with no surface of the indent forming part of the proximal or distal surface of the segment.

Also, to the extent that there is some adherence between the bending section material and the glue anchor 15, increasing the contact area between the two, which is also done by the ledge 16, will improve the stability of the anchor position.

For the glued anchor, the indent may be placed in a segment with the segment limiting the indent both in the distal and in the proximal direction. This may make it simpler to control where the glue is flowing during the gluing process. The indent may, therefore, be molded in an intermediate area of a segment rather than on a proximal or distal side thereof, as shown in FIG. 5, where a segment 22a is longer than the segments 22 and includes the indent 14 between its proximal and distal ends. The same is shown in FIG. 6, where the segment 22a is sectioned across the central ledge portion 16′ to better show the anchors 15.

It should be noted that the bending section 20 may often be provided with a bending cover 21 on the external side. This provides a watertight barrier towards the surroundings of the endoscope insertion cord 3. This bending cover 21 may have some effect on supporting the anchor 15 in the correct position. The effect may, however, be relatively small as the bending cover is often very flexible and not glued to the bending section except perhaps at the proximal and distal ends thereof.

For the glue anchor a UV curing glue may be applied, as curing can happen immediately, and thereby limiting the time for the glue to flow into unwanted places inside the bending section. The glue for the anchor 15 may be applied from the outside of the bending section 20, e.g., using a small needle tip for precise application. Viscosity of the glue may be selected to be low enough for the glue to flow in between the steering wire 25 and the working channel tube 6′ to form the holding part 17. Also, the viscosity of the glue may be selected to be high enough to avoid glue flowing into unwanted parts of the bending section via capillary forces. Also, it may be easier to create a smooth surface on the outside of the anchor 15, i.e., the external side of the bending section, with a higher viscosity glue with higher surface tension.

As indicated above, one way to make the anchor 15 is to first insert the working channel tube 6′ and the wire coils 27 in the bending section and then apply the glue or adhesive so that it can form the holding part 17. To make the indents 14 in an injection molding process, a mandrel is positioned inside a, for example, two-part mold. The two-part mold includes surfaces to form the outer wall surface 23′ and protrusions extending perpendicularly therefrom into the cavity formed by the two-part mold. Some of the perpendicular protrusions form the gaps between segments that define the hinges and some of the perpendicular protrusions form the indents. A protrusion can also form the segment gaps and provide indents on the proximal and/or distal surfaces of the adjacent segments. The mandrel is mostly cylindrical but has a longitudinal protrusion for forming the electrical wire bundle channel 7′ and two longitudinal protrusion for forming the grooves for the steering wires 25 and/or the wire coils 27. After the molten polymer is injected into the cavity and solidified, the mandrel is pulled out from the formed bending section and the two sides of the two-part mold are pulled out, removing the protrusions from the formed indents 14 and from the gaps between the segments.

Two anchors can be positioned at a same longitudinal position in the bending section. Two anchors can also be positioned in different longitudinal positions in the bending section. The indents for the anchors can be placed together with or independently of the segment gaps. FIG. 6C shows a bending section from two sides. A proximal section 20a of the bending section 20 extends from the proximal end, and a distal section 20b extends from the proximal section 20a to the distal end. The lower view shows the same bending section as the upper view but the bending section has been rotated about its longitudinal axis 180 degrees. Nearest the proximal end are two anchors 15 positioned on opposite sides of a plane traversing a hinge and at the same longitudinal position. Further from the proximal end are two anchors 15 positioned on opposite sides of the plane but at different longitudinal positions. These anchors are positioned in indents that replace segment gaps. To this end the mandrel has an indent making protrusion intermediate two segment gaps. The length of the bending section includes the proximal and distal ends. The proximal section 20a may be, preferably, 60% of the bending section length. The anchors are preferably positioned in the proximal section 20a.

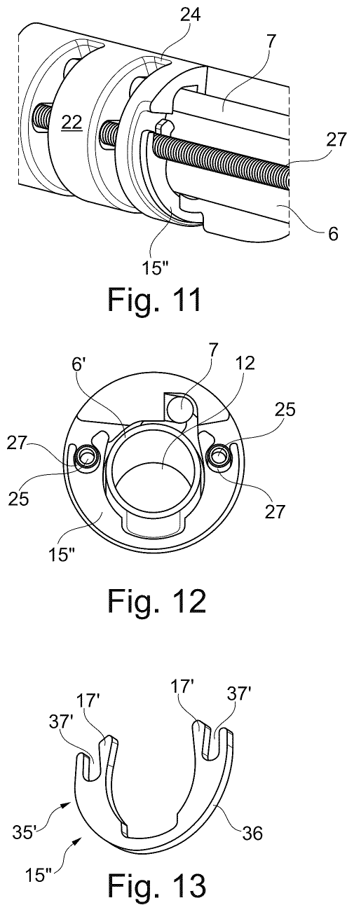

FIGS. 7-12 show an embodiment where the anchor 15 is made from a disc 35. The shape of the disc may be like the Greek letter omega, of horse-shoe shaped, or U-shaped. This disc is arranged in an indent 14, where the indent may be in a segment 22 as shown in FIGS. 10 and 11, or the indent may be in a hinge 24 connecting two segments 22, as shown in FIGS. 7 and 9. Preferably, the disc 35 may comprise an anchor part 15 for each of the two steering wires, or, alternatively, the disc 35 may only have an anchor part for one of the steering wires 25.

The disc 35 is preferably made from a hard material, e.g., a metal such as steel. The disc may also be made from a relatively hard polymer, e.g., polycarbonate (PC), polystyrene (PS), or polyamide (PA). The thickness of the disc may depend on the material. For steel the thickness may be in the range 0.1-0.5 mm, preferably 0.1-0.3 mm. If the material is a polymer the thickness may be above 0.3 mm.

The disc 35 may be shaped so that an outer circumference 36 of the disc is level with an outer surface formed by the parts of the bending section segments 22 and the hinges 24 facing opposite to a central passage 12 of the bending section 20. This outer surface may also in this embodiment be covered by a bending cover 21.

The disc 35 is provided with a holding part 17′ extending into the space between the steering wire 25 and the working channel tube 6′ such that displacement of the steering wire 25, and maybe also the spring 27, in a direction towards the working channel 6, or a center of the central passage, is prevented.

When the disc 35 is arranged in an indent 14 in a hinge 24, this may limit the angle by which the two neighboring segments, being next to this hinge, may be able to bend relative to each other. For some applications of the endoscope such a limitation may be acceptable. If the bending section is provided with many hinges, e.g., 20 or more, this limited bendability of maybe two pair of hinges might not affect the overall bending performance significantly.

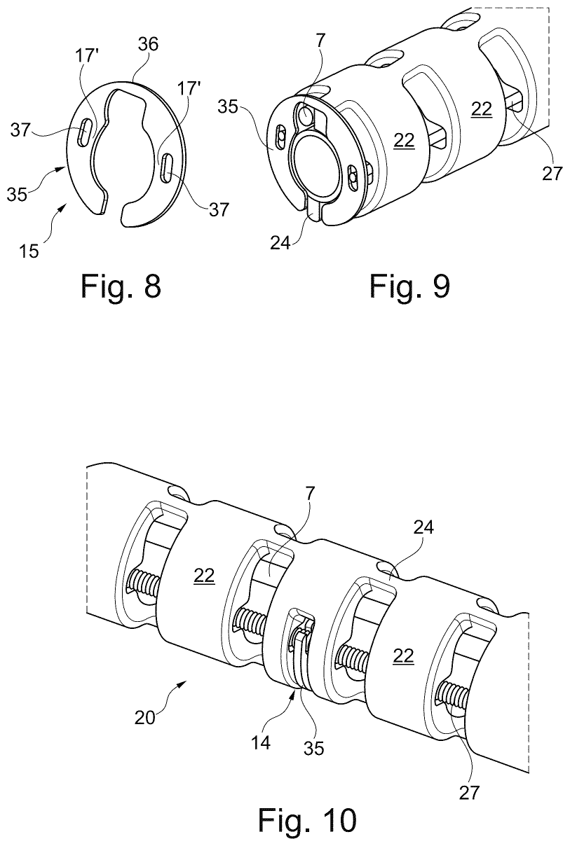

FIGS. 8 and 9 shows a variation of the embodiment where the anchor 15′ is a disc 35. In this variation passages 37 for the steering wires, and e.g., also for the springs, are provided as closed holes. This means that the steering wires and e.g., the springs will need to be threaded through these holes.

Alternatively, another variation of the anchor 15″ provided as a disc 35, is shown in FIGS. 10-13. In this variation the passages 37′ are not closed, but open at one side, so that the disc 35 can be inserted into the indent 14 after the steering wires 25, and e.g., the springs 27, have been arranged in the grooves for the steering wires 25, and e.g., the springs 27. In this variation the indents are preferably made in a segment 22 of the bending section.

It is a possible to combine different types of anchors at different positions in the same bending section. The anchors could, in general, be the examples provided above, but it could also be other types or designs of anchors.

The number and positions of anchors along a proximal-distal direction of the bending section may depend on the total length of the bending section, the outer diameter of the bending section, the maximum bending angle of the distal end of the bending section relative to the proximal end of the bending section, and on the necessary pulling force on the steering wires to obtain this bending angle.

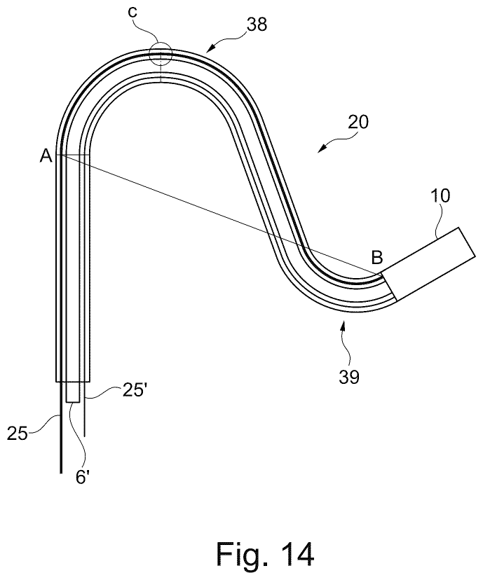

For example, an ureteroscope may have a bending section with a length of more than 35 mm, maybe more than 45 mm. The outer diameter may be around 3 mm or below 3 mm. The maximum bending angle may be around 270 degrees, maybe in the range 225-270 degrees. When a ureteroscope is used in a procedure in a kidney, it may often happen that the bending section has a first curvature 38 in the proximal half length of the bending section going in one direction, and at the same time needs to bend the distal end of the bending section in the opposite direction, resulting in a second curvature 39. This is illustrated schematically in FIG. 14, where a resulting S-shaped curvature of the bending section can be seen.

This means that there will be a relatively high force on the steering wire 25 to the convex side of the first curvature 38 or proximal bending curve, as this steering wire 25 needs to be pulled to bend the distal end of the bending section into the second curvature 39. This force is directed towards a center of the central passage of the bending section. In general, the force on the steering wire 25 will tend to move it towards the line A-B. The circle c indicates the area where the force towards the line A-B is maximum for the steering wire 25. Therefore, anchors 15, 15′, 15″ may be placed in the part of the bending section where this area with the maximum force is likely to be during a procedure.

Looking at the bending of the bending section shown in FIG. 14, it is realized that the other steering wire 25′ placed to the concave side of the first curvature 38, will be affected by a relatively smaller force, since this steering wire does not have to be pulled to bend the distal end of the bending section into the second curvature 39. Also, the force on the other steering wire 25′ in the first curvature 38 is directed towards the groove 18 and the circumferential wall 23 of the segments 22, which will be more resistant towards any unintended displacement of the steering wire compared to the working channel 6.

The above-mentioned embodiments or variations of the anchor, and how the anchor is arranged and connected to the bending section, are not limiting for the scope of this disclosure. Other designs, arrangements and connections of the anchor are possible within the scope of the claims.

The following items are further variations and examples of the embodiments described with reference to the figures.
1. An endoscope comprising: a handle; an insertion cord extending from the handle and including a bending section molded in one piece, the bending section comprising: segments connected to each other by hinges, the hinges allowing two neighboring segments to bend relative to each other in a bending plane, the segments and the hinges form an outer surface of the bending section, a central passage passing each segment and extending from a distal end to a proximal end of the bending section, where each segment has a circumferential wall, the circumferential wall has an inner surface facing and encircling the central passage, at least two grooves extending from the inner surface into the circumferential wall, the grooves extending in parallel with the central passage and in open connection with the central passage, the central passage and the grooves extending through the segments of the bending section, an indent extending from the outer surface into at least one segment or in at least one hinge; two steering wires extending from the handle to the distal end of the bending section, each steering wire passes in a respective one of the grooves through the bending section; wherein an anchor is arranged in the indent, the anchor being provided with a holding part such that displacement of the steering wires from the grooves towards a center of the central passage is blocked.
2. The endoscope according to claim 1, wherein anchors are placed in at least two indents in segments, or next to segments, placed in the 60% of the bending section length extending from the proximal end of the bending section towards the distal end.
3. The endoscope according to claim 2, wherein at least two segments are provided with anchors such that displacement of the two steering wires is blocked.
4. The endoscope according to claim 2 or 3, wherein at least two segments provided with anchors are separated by at least one segment without an anchor.
5. The endoscope according to claim 1, wherein each anchor has a holding part extending into a space where the groove and the central passage meet.
6. The endoscope according to claim 1, wherein the steering wires in the bending section are passing inside coil springs, the coil springs with the steering wires are each arranged in a groove, and the anchor is arranged to block displacement of the coil springs, and thereby the steering wires, from the grooves towards a center of the central passage.
7. The endoscope according to claim 1, wherein the anchors having an exterior surface being level with an outer surface of a segment and the anchors having an interior surface being level with the inner surface.
8. The endoscope according to claim 1, wherein the anchors are made from hardened glue.
9. The endoscope according to claim 8, wherein a ledge is extending from the segment between the indent and the central passage, the ledge is supporting the anchor.
10. The endoscope according to claim 1, wherein the anchors are made from a metal disc.
11. The endoscope according to claim 1, wherein anchors are arranged in at least two segments, and in less than 40% of the segments, alternatively in less than 30% of the segments.
12. A method for assembling an endoscope according to any one of the previous claims, comprising: providing a bending section molded in one piece from a fused polymer, and having: segments connected to each other by hinge members, the hinges allowing two neighboring segments to bend relative to each other in a bending plane, the segments and the hinges forms an outer surface of the bending section, a central passage passing each segment and extending from a distal end to a proximal end of the bending section, where each segment has a circumferential wall, the circumferential wall has an inner surface facing and encircling the central passage, at least two grooves extending from the inner surface into the circumferential wall, the grooves extending in parallel with the central passage and in open connection with the central passage, the central passage and the grooves extending through the segments of the bending section, an indent extending from the outer surface into at least one segment or in at least one hinge; arranging two steering wires to extend from the handle to the distal end of the bending section, such that each steering wire passes in a respective one of the grooves through the bending section; arranging an anchor in the indent, where the anchor is provided with a holding part such that displacement of the steering wires from the grooves towards a center of the central passage is blocked.
13. A system comprising an endoscope according to any one of claims 1-11, a monitor and a control unit.

1. An endoscope comprising:
a handle;
an insertion cord extending from the handle and including a bending section, the bending section comprising a one-piece part including a distal end and a proximal end;
a first steering wire extending from the handle to the distal end of the bending section; and
a second steering wire extending from the handle to the distal end of the bending section,
the bending section comprising:
segments connected to each other by hinges, the segments and the hinges forming an outer surface of the bending section,
a central passage extending from the distal end to the proximal end, each segment including a circumferential wall comprising an inner surface facing and encircling the central passage,
grooves extending from the inner surface into the circumferential wall of each segment and in open connection with the central passage,
an indent extending from the outer surface into a segment or a hinge, the indent positioned intermediate the proximal end and the distal end, and
an anchor comprising a holding part,
wherein the first steering wire passes through the grooves of each segment through the bending section, and
wherein the anchor is arranged in the indent with the holding part blocking displacement towards a center of the central passage of a portion of the first steering wire transversely adjacent the anchor.

2. The endoscope of claim 1, wherein the endoscope consists of an ureteroscope or a cystoscope, the endoscope further comprising a working channel tube in the central passage, and a distal tip including a camera, an outer diameter of the bending section being between 2.0 and 3.0 mm.

3. The endoscope of claim 1, wherein the endoscope consists of an ureteroscope or a cystoscope, the endoscope further comprising a working channel tube in the central passage, a distal tip including a camera, and a bending sleeve over the bending section, an outer diameter of the bending sleeve being between 2.0 and 3.0 mm, wherein a cross-sectional area of the working channel tube is at least 40% of a cross-sectional area of the bending section.

4. The endoscope of claim 1, the endoscope further comprising a working channel tube in the central passage and a first coil spring surrounding the first steering wire within the bending section, wherein the first coil spring passes through the grooves, wherein the holding part extends into a space where the groove and the central passage meet, wherein the holding part contacts and is adhered to a portion of the first coil spring, and wherein the anchor is arranged in the indent with the holding part blocking displacement towards the center of the central passage of the portion of the first coil spring contacting the holding part.

5. The endoscope of claim 4, wherein the two indents are provided in two of the segments, and wherein the two of the segments are separated by at least another one of the segments, the at least another one of the segments not being provided with an indent.

6. The endoscope of claim 1, the endoscope further comprising a working channel tube in the central passage and a first coil spring surrounding the first steering wire within the bending section, wherein the bending section comprises two anchors located at the same longitudinal position of the bending section on opposite sides of a bending plane, the two anchors including the anchor, wherein the bending section comprises two additional anchors located at the same longitudinal position of the bending section on opposite sides of a bending plane, the two additional anchors located distally of the two anchors with at least two segments between them, wherein the bending section comprises a proximal section extending from the proximal end toward the distal end and comprising 60% of a length of the bending section, wherein the two anchors and the two additional anchors comprise glue anchors positioned in the proximal section of the bending section.

7. The endoscope of claim 1, wherein the bending section comprises two anchors and two indents located at different longitudinal positions of the bending section, wherein the two anchors comprise the anchor and a second anchor and the two indents comprise the indent and a second indent, wherein the bending section comprises a proximal section extending from the proximal end toward the distal end and comprising 60% of a length of the bending section, and wherein the two indents are positioned in the proximal section of the bending section.

8. The endoscope of claim 1, wherein the holding part extends into a space where the groove and the central passage meet.

9. The endoscope of claim 1, wherein the holding part overlaps, longitudinally, the groove between the first steering wire and the inner surface of the central passage.

10. The endoscope of claim 9, further comprising a first coil spring surrounding the first steering wire within the bending section, wherein the first coil spring passes through the grooves, wherein the holding part contacts a portion of the first coil spring, and wherein the anchor is arranged in the indent with the holding part blocking displacement of the portion of the first coil spring contacting the holding part towards the center of the central passage.

11. The endoscope of claim 1, wherein the anchor comprises an exterior surface and an interior surface, the exterior surface being level with the outer surface of the bending section and the interior surface being level with the inner surface of at least one segment.

12. The endoscope of claim 1, wherein the anchor is a glue anchor comprised of hardened glue, the segment comprising a ledge central portion and ledges extending from the ledge central portion, the ledges and the ledge central portion comprising an inner surface adjacent the central passage and each of the ledges comprising an opposite surface, the indent being located between the opposite surfaces of the ledges and the outer surface of the bending section.

13. The endoscope of claim 1, wherein the anchor is a glue anchor comprised of hardened glue, wherein the segment comprises a cut-out between a proximal surface and a distal surface of the segment, the indent being formed by the cut-out with no surface of the indent being part of the proximal surface or the distal surface of the segment.

14. The endoscope of claim 1, wherein the anchor is a glue anchor comprised of hardened glue, wherein the segment comprises a cut-out between a proximal surface and a distal surface of the segment, the indent being formed by the cut-out with a surface of the indent being part of the proximal surface or the distal surface of the segment.

15. The endoscope of claim 1, wherein the anchor is a glue anchor comprised of hardened glue.

16. The endoscope of claim 1, wherein the anchor is a glue anchor comprised of hardened glue, the bending section further comprising a ledge having an inner surface adjacent the central passage and an opposite surface, the indent being located between the opposite surface of the ledge and the outer surface of the bending section.

17. The endoscope of claim 16, the bending section further comprising a ledge between the indent and the central passage, wherein the ledge supports the glue anchor.

18. The endoscope of claim 1, wherein the anchor is an insert anchor inserted into the indent, the bending section further comprising a ledge between the indent and the central passage, wherein the ledge supports the insert anchor.

19. The endoscope of claim 1, wherein the anchor consists of a metal disc.

20. The endoscope of claim 1, further comprising a metal cylinder positioned in one of the grooves, wherein the first steering wire passes through the metal cylinder, wherein the anchor consists of a glue anchor, and wherein the holding part is adhered to the metal cylinder.

21. The endoscope of claim 1, the endoscope further comprising a working channel tube in the central passage, wherein the anchor is one of a plurality of anchors positioned in 10% to 30% of the segments within a proximal section of the bending section, the proximal section comprising less than 60% of a length of the bending section, wherein the anchors are spaced so that there is at least one anchorless segment between segments with anchors of the plurality of anchors.

22. A system comprising the endoscope of claim 1, a monitor and a control unit.
